(19) United States
(12) Patent Application Publication
(10) Pub. No.: US 2024/0148299 A1
(43) Pub. Date: May 9, 2024

(54) INGESTIBLE ANXIETY MONITORING AND TREATMENT SYSTEM

(71) Applicant: CELERO SYSTEMS, INC., Lincoln, MA (US)

(72) Inventors: Benjamin D. Pless, Lincoln, MA (US); Daniel Bacher, Lincoln, MA (US); Shannon Schuetz, Lincoln, MA (US)

(21) Appl. No.: 18/549,953

(22) PCT Filed: Mar. 14, 2022
(86) PCT No.: PCT/US22/20112

§ 371 Date: Sep. 11, 2023

Related U.S. Application Data
(60) Provisional application No. 63/160,015, filed on Mar. 12, 2021.

(51) Int. Cl. A61B 5/16; A61B 5/00; A61B 5/053; A61B 5/07

(52) U.S. Cl. CPC A61B 5/165; A61B 5/053; A61B 5/073; A61B 5/4839; A61B 5/6861; A61B 2562/162

(57) ABSTRACT

An anxiety monitor and treatment system is provided comprising an ingestible housing and at least one sensor configured to detect a physiological parameter indicative of anxiety. The system further includes a controller operatively coupled to the least one sensor. The controller is programmed to receive the physiological parameter and prepare the physiological parameter for transmission from the subject, analysis of the physiological parameter, or both.

### CROSS-REFERENCE TO RELATED APPLICATIONS

The present application claims priority to U.S. Provisional Application No. 63/160,015, filed on Mar. 12, 2021, and which is incorporated by reference herein.

### TECHNICAL FIELD

The present application relates to an ingestible device and system to monitor and optionally treat anxiety.

### BACKGROUND

Experiencing occasional anxiety is a normal part of life. However, individuals with anxiety disorders frequently experience intense, excessive and persistent worry and fear about everyday situations. Often, anxiety disorders involve repeat episodes of sudden feelings of intense anxiety and fear or terror that reach a peak within minutes (e.g. panic attacks). Panic disorder is fairly common and affects about 6 million adults in the U.S. These feelings of anxiety and panic can interfere with daily activities, can be difficult to control, can be out of proportion to the actual danger, and can last a long time. For example, patients may avoid places or situations to prevent these feelings. Symptoms may start during childhood or the teen years and continue into adulthood.

### SUMMARY

The present invention provides a simple, unobtrusive and convenient platform with which to detect whether a panic attack or other form of anxiety is imminent, and either delivers an anti-anxiety medication or alerts the patient to take a fast-acting medication such as a benzodiazepine or other anti-anxiety medication.

In an aspect, an anxiety monitor and treatment system is provided. The system comprises
an ingestible housing, at least one sensor configured to detect a physiological parameter indicative of anxiety, and a controller operatively coupled to the least one sensor. The controller is programmed to receive the physiological parameter and prepare the physiological parameter for transmission from the subject, analysis of the physiological parameter, or both.

### BRIEF DESCRIPTION OF DRAWINGS

FIG. 1A is a block diagram of an exemplary anxiety monitor and treatment device according to an aspect of the present disclosure.

FIG. 1B is a block diagram of an exemplary anxiety monitor and treatment device according to an aspect of the present disclosure.

FIG. 2A is a perspective view of an exemplary anxiety monitor and treatment device according to an aspect of the present disclosure and FIG. 2B is an inverted view of FIG. 2A.

FIG. 3 is a top plan view of an exemplary anxiety monitor and treatment device where the according to an aspect of the present disclosure.

FIG. 4 is side view of an exemplary anxiety monitor and treatment device according to an aspect of the present disclosure.

FIG. 5 is a perspective view of an exemplary anxiety monitor and treatment device according to an aspect of the present disclosure.

### DETAILED DESCRIPTION

As used herein with respect to a described element, the terms “a,” “an,” and “the” include at least one or more of the described elements including combinations thereof unless otherwise indicated. Further, the terms “or” and “and” refer to “and/or” and combinations thereof unless otherwise indicated. By “substantially” is meant that the shape or configuration of the described element need not have the mathematically exact described shape or configuration of the described element but can have a shape or configuration that is recognizable by one skilled in the art as generally or approximately having the described shape or configuration of the described element. A “patient” as described herein includes a mammal, such as a human being. An “ingestible housing” as used herein is a housing that is not just capable of being ingested but rather is suitable and intended for swallowing and entering into the gastrointestinal tract for therapeutic purposes. By “mitigating the effects of anxiety in a patient” means mitigating the adverse consequences of anxiety in a patient whose condition improves compared to the patient's condition prior to the anxiety. An “anxiety monitor and treatment device” can include a device that just monitors anxiety or that monitors and treats anxiety.

In an aspect, an anxiety monitor and treatment system is provided. As described in more detail below, the system comprises an ingestible housing, at least one sensor configured to detect a physiological parameter indicative of anxiety, and a controller operatively coupled to the least one sensor. The controller is programmed to receive the physiological parameter and prepare the physiological parameter for transmission from the subject, analysis of the physiological parameter, or both.

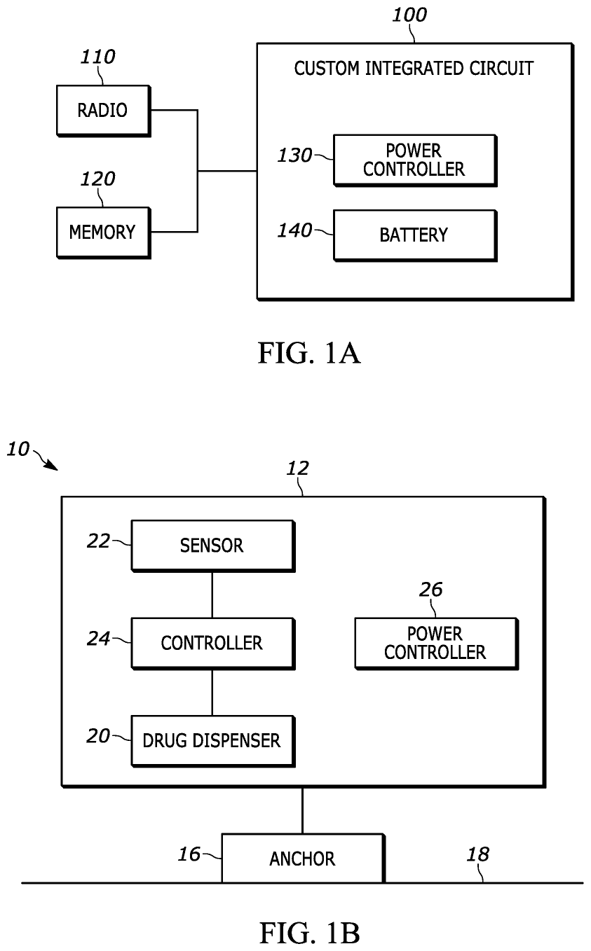

In particular, an anxiety monitor and treatment device is provided that contains an electronic circuit contained in a small housing, such as a polycarbonate housing, suitable for swallowing and for monitoring as well as treating anxiety. Referring to FIG. 1A, a representative block diagram of an anxiety monitor and treatment device is shown. Once ingested and reaching, for example, the stomach, the device can monitor physiological parameters indicative of anxiety such as ECG, respiration, saturated oxygen, and core temperature. Most of the functionality can be contained in a custom integrated circuit 100 which can contain a power controller 130 that distributes power from a battery 140 to the rest of the circuits (not shown). The custom integrated circuit 100 can route the output of the rest of the circuits (not shown) for storage in memory 120. For example, an accelerometer and gyroscope circuit (not pictured) can be sampled to measure user activity levels and respiration based on movement of the abdomen. The present disclosure also provides for a system that can include a wearable flexible sensor for determining swear analyte, for example. Upon detecting physiological indications of an anxiety attack, the system can send out alerts and/or release an anti-anxiety medication such as a benzodiazepine, for example.

In more detail and with reference to FIG. 1B, in an embodiment, an anxiety monitor and treatment device 10 is provided that includes an ingestible housing 12. An anchor 16 can be connected to the distal end of housing 12 and can be sized and configured to retain the housing in the stomach and prevent premature passage of the device through the pylorus into the intestine. Device 10 can also include a drug dispenser 20, such as a non-refillable drug dispenser, that contains an anti-anxiety medication. In embodiments where the drug dispenser is non-refillable, it can be a reservoir that does not include any ports or similar structures that allow the drug dispenser to be re-filled from a location external to the patient's body, such as a syringe containing an anti-anxiety medication that is injected into the drug dispenser from outside the patient's body. Device 10 can also include at least one sensor 22 contained within housing 12 that is configured to detect at least one physiological parameter indicative of anxiety. Device 10 can also comprise controller 24 operatively coupled to drug dispenser 20 (in embodiments including a drug dispenser) and sensor 22. Controller 24 can be configured to receive a signal detected by sensor 22 of the physiological parameter to actuate release of the anti-anxiety medication from drug dispenser 20 into the patient's stomach. Device 10 can also include a power controller 26 electrically connected to controller 24.

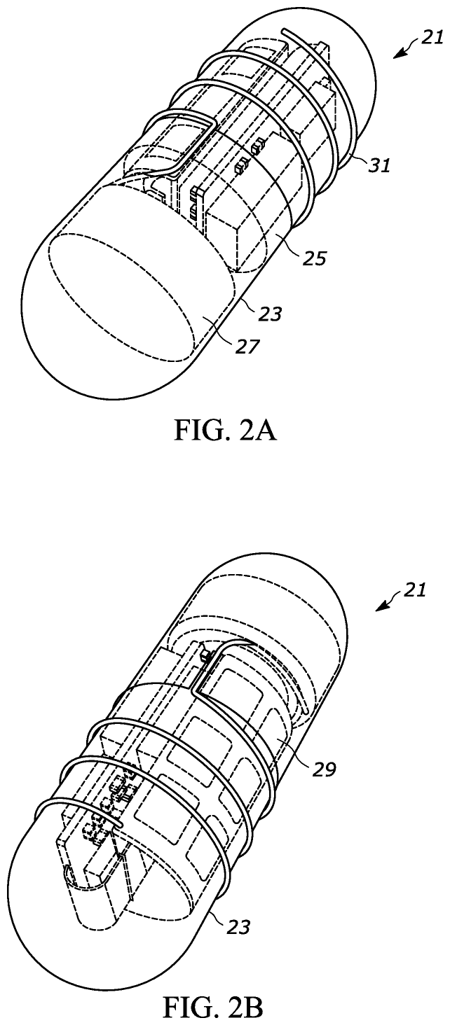

FIGS. 2A and 2B illustrate an exemplary arrangement of certain components of an anxiety monitoring device 21. For example, device 21 can comprise a housing 23 within which is contained a controller 25, a power controller in the form of a battery 27, and an optional drug dispenser 29. Disposed about the outer surface of housing 23 is an antenna 31. The device can comprise other components as described below. As stated above, the ingestible housing can be a gelatin housing or can have another type of suitable enteric coating. The housing can have any suitable size for swallowing such as between size 000 to size 4 and can also have any suitable configuration such as, for example, a capsule or tablet.

As stated above, at least one sensor contained with the housing is configured to detect at least one physiological parameter indicative of anxiety. Such sensors can measure ECG, respiration, saturated oxygen, and core temperature, for example. Further, a controller can be contained within the interior of the housing and be operatively coupled to the drug dispenser and the least one sensor. The controller can be configured to receive a signal detected by the least one sensor of the at least one physiological parameter to actuate release of the anti-anxiety medication from the drug dispenser into the gastrointestinal tract of the patient in response to a determination that the at least one physiological parameter falls outside a threshold value or range for the at least one physiological parameter or meets pre-defined conditions indicating that anxiety has been detected.

For example, by monitoring respiration, heart rate, activity, core temperature, saturated oxygen levels, etc. the device can detect the presence of physiologic distress when individual physiological parameters, trends in physiological parameters or combinations of physiological parameters meet predefined conditions. For example, a trend towards abnormal breathing (e.g. fast or irregular) and an increased heart rate and sweating, not associated with normal activity or exercise, could trigger the ingested device to automatically release a fast-acting medication such as a benzodiazepine to thwart the panic attack early. Alternatively, if the device detects physiologic distress, the ingested device could send a message to a smart phone carried by the patient, or caregiver, to alert the patient to either a) take an oral fast-acting anxiety medication or b) authorize/confirm that the fast-acting anxiety medication should be released from the ingested device. Lastly, the ingested device could also release an anti-anxiety medication according to a prescribed schedule to increase medication compliance.

In certain aspects, a sensor is an accelerometer that can detect motion of the abdomen during respiratory inhalation and exhalation as well as measuring other movement signals generated by the patient moving around (e.g. walking, coughing, or other physical activity). The sensor can be a 3-axis accelerometer, measuring linear acceleration in 3 dimensions. Inside the body, the accelerometer can pick up any full body movements (e.g. acceleration), but when the patient's body is at rest, one of the motions that is still present is movement of the gut during breathing (e.g. inhalation and exhalation cause movement of the abdomen and therefore movement of the device). Hence suitable signal processing (e.g. filtering) can be applied to the accelerometer signals to isolate movement signal caused by respiration and thereby estimate respiratory rate. There are other types of motion in the GI tract like peristalsis that can be detected by the accelerometer, and certain cardiac ballistic activity (e.g. slight movements caused by blood flowing each beat of the heart) as well. The accelerometer signal processing can operate in parallel with signals obtained by the PPG sensor as well, and a combination of estimates can provide more accurate estimates of respiratory rate along with SaO2.

In addition or in alternative to the physiological sensors mentioned herein, a system can also be provided that includes wearable sensors. Such wearable sensors may include wearable flexible sensors that measure, for example, heart rate, body temperature, levels of blood sugar, metabolic byproducts, sweat analytes, or combinations thereof. Further, systems can include a galvanic skin sensor to measure electrodermal activity (e.g. electrical conductance of the skin). Further, systems can include a transcutaneous electrode that would deliver therapeutic electrical stimulation in response to the detected physiological parameter(s).

As mentioned above, a non-refillable drug dispenser can be contained within the housing of the anxiety monitor and treatment device and comprise an anti-anxiety medication. The drug dispenser can contain any suitable anti-anxiety medication including a plurality of anti-anxiety medications. The drug dispenser can be loaded in the housing and can include more than one drug dispenser, with each dispenser containing the same drug, different concentrations or release characteristics (e.g. extended, fast) of the same drug, or different drugs. Several types of medications can be used to help relieve symptoms, depending on the type of anxiety disorder. For example, certain antidepressants can be used to treat anxiety disorders and certain anti-anxiety medications such as buspirone can also be used. In certain circumstances, other types of medications, such as sedatives, also called benzodiazepines, or beta blockers can be used. These medications can be used for short-term relief of anxiety symptoms.

While many schedules of anti-anxiety medication release are possible, an example would be an initial automatic release by the device of 0.5 mg of lorazepam, or other benzodiazepine, followed by monitoring for 90 seconds. If physiological parameters are unchanged or worsen, an additional 0.5 mg of lorazepam could be automatically released followed by an additional monitoring period of 90 seconds. That cycle could continue until a total of 10 mg of lorazepam had been released or physiological parameters improved. Throughout the delivery of therapy, the device could store samples of physiological signals in its memory and log the time of drug release and data storage. The stored information could be uploaded automatically along with alerts to the patient's cell phone for subsequent uploading to a centralized database for review by authorized clinicians. Alternatively, doses of 2 mg of lorazepam, for example, could be delivered with the controller being configured to deliver multiple doses. Such dosages are exemplary and other doses and dosing schedules could be employed.

In another embodiment of this invention, the device can be configured to deliver lorazepam, for example directly in the stomach based on a programmed schedule. For instance, at set intervals spanning hours or days the ingestible device could release small doses of lorazepam to prolong the pharmacological effect of the anti-anxiety medication. Intervals and doses can be programmed by a clinician customized to the needs of the patient. In this embodiment, the device may also deliver an initial larger anti-anxiety medication dose (e.g. 5+mg) if detected by the physiological sensors, and then subsequently continue release of smaller doses of the medication over an extended period of time (many hours or days) to protect the individual from subsequent overdose events. Upon depletion of the medication, a wireless command can be sent to the smartphone to inform the user and/or clinician that a new ingested device would be required to continue delivery of the anti-anxiety medication if clinically desired.

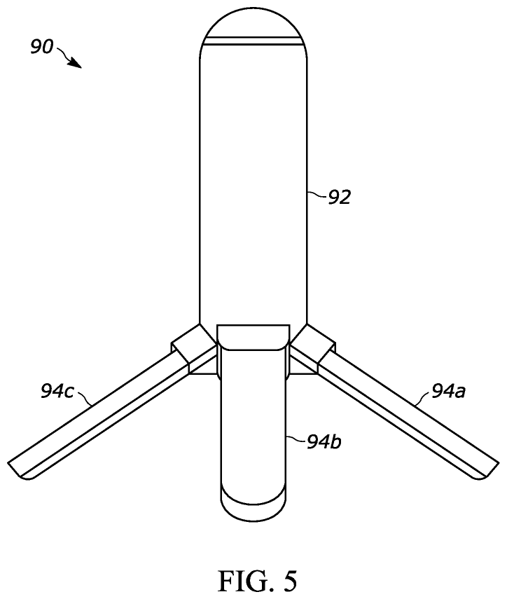

In certain aspects, the device resides in the patient's stomach for a temporary period of time without passing through the pylorus into the intestine. For example and with reference to FIG. 5, an anxiety monitor and treatment device 90 can include an ingestible housing 92 having detachable residency arms 94 extending from the distal end thereof that are sized and configured to retain the device in the stomach until the desired monitoring/treatment has occurred. The detachable residency arms are also configured to detach and pass through the GI tract after such monitoring/treatment has occurred. In other aspects, upon being ingested, the housing can pass through the patient's stomach and dissolve in the intestine thereby releasing the electronic circuit. As mentioned above, sensors within the housing can monitor one or more physiological parameters indicative of anxiety such as electrical cardiac activity, heart rate, heart rate variability, respiratory monitoring (e.g. respiratory rate), saturated oxygen, intestinal tissue color, central temperature, bodily motions as detected from the intestine, or combinations thereof. Physiological parameters can be measured by an electrocardiogram (ECG) sensor, an accelerometer, a photoplethysograph (PPG) sensor, a temperature sensor, or combinations thereof. In response to detecting physiological indications of anxiety, the ingested device can release an anti-anxiety medication via a drug dispenser as well as send out alerts to the patient and/or a caregiver. Non limiting examples of suitable drug dispensers include MEMS drug delivery, valve systems, osmotic plug pistons, electrolytical pumps, or combinations thereof.

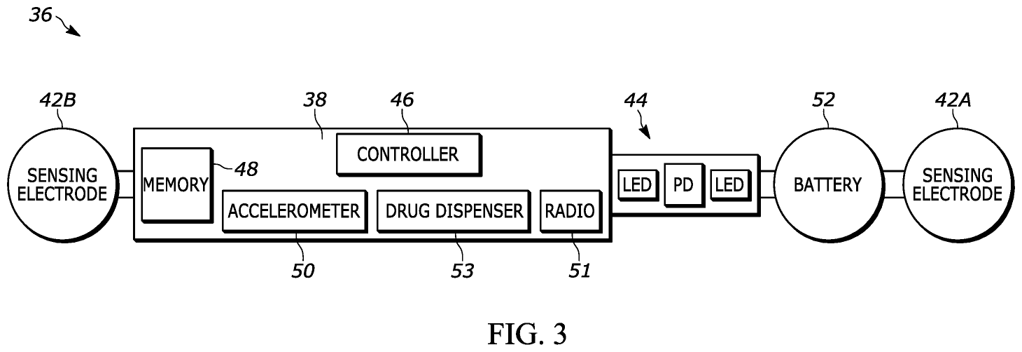

FIG. 3 is a top plan view of an exemplary anxiety monitor and treatment device 36. In this particular aspect, the anxiety monitor and treatment device comprises a PPG sensor 44, an accelerometer 50, and sensing electrodes 42A and 42B that can be used for ECG monitoring and/or for impedance plethysmography (IPG). However, additional or fewer sensors could be used so long as at least one physiological parameter indicative of anxiety is measured. Device 36 can also include a controller 46, memory 48, a battery 52, a drug dispenser 53, a non-specific radio module, or other wireless communication module 51. The controller, the PPG sensor, and the accelerometer can be formed at least partially as an application specific integrated circuit (ASIC).

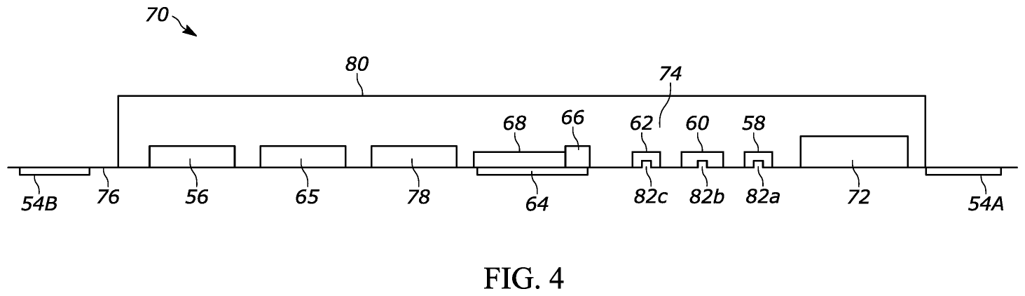

Referring to FIG. 4, in this aspect, an anxiety monitoring/treatment device 70 can include ECG/plethysmography electrodes 54A and 54B. A circuit ground electrode 64 can also be included. Other components including memory 56, controller 68, communication module 78, a battery bypass capacitor 66, and PPG LEDs 62 and 58 and photodiode 60 can be located on top side 80 of interior of housing 74. Drug dispenser 65 can be located on either the top or bottom surface so long as drug can be released from the dispenser and absorbed into the stomach (in embodiments including a drug dispenser). A battery 72 can power device 70. While the pulse PPG components 58, 60, and 62 are illustrated as being on top side 80 they can be directed through apertures 82 towards bottom side 76 which is where the mucosa of the stomach will be.

Electrodes can connect to an ECG amplifier and filter, and to a plethysmogram circuit. A ground electrode can provide a reference voltage to the patient and can be connected to a power controller that distributes power from a battery to the rest of the circuits. The power controller can also control the connectivity of a battery bypass capacitor to reduce power consumption before the circuit is activated. The ECG amplifier can provide a gain of about 200 and can be band pass filtered between about 2 Hz and 100 Hz. The plethysmogram can inject a series of (for example) 50 uA current pulses approximately 30 μsec in duration at a rate of about 30 Hz into the electrodes. A synchronous demodulator can construct an envelope of the impedance waveform from the voltage resulting from the current pulses. From the impedance signal, a respiration signal can be derived. The controller can route the output of the plethysmogram or the ECG amplifier to an A to D converter (ADC) for subsequent storage in a memory, such as static RAM (SRAM), for example.

The device can have a crystal controlled real-time clock which can be used for a number of functions including time stamping events and logs stored in the memory. In addition, using the plethysmogram circuit, the device can monitor the impedance across the electrodes and automatically detect when the device has reached the target site, such as the stomach. At a fixed time later (for example at two weeks), the service life of the device can be programmed to come to an end. As mentioned above, the device can automatically initiate a process to cause mechanical links between circuit elements to break down. This can be done, for example, by energizing biogalvanically corrodible links between the circuit elements. In an alternative embodiment, the biodegradable links can be engineered to be biostable for a period of time (e.g. 10 to 14 days) and then rapidly biodegrade.

In addition to storing the activation time in memory, other event logs may be stored. For example, the controller can analyze the ECG and store the time and date of cardiac events such as tachycardia and bradyarrhythmias. The controller can also analyze the plethysmogram and store the date and time of respiratory events such as apnea, or disordered breathing. In addition, a PPG circuit can work in conjunction with infrared and red LEDS and a photodiode to monitor saturated oxygen (SaO2). The controller can detect changes in SA02, in particular drops of 3% or greater, and store the date and time of these events in the memory. In addition, the PPG signal can be analyzed by the controller to derive a respiration signal. The PPG signals can also be analyzed by the controller to derive relative change in intestinal wall color. An integrated temperature circuit can be sampled by the controller on a regular basis for storage in the memory. In addition to the physiological sensors mentioned above, additional sensors may be used, such as, for example, a microphone, or a pressure sensor.

A Bluetooth radio can communicate directly with a smart phone or other Bluetooth enabled device outside the patient's body. The Bluetooth radio can be a 2.4 GHz Bluetooth or a BLE (Bluetooth Low Energy) radio. The communication link can allow a physician to configure the ingested device to collect certain data and to retrieve the data after it has been collected. In an alternative configuration, wireless data telemetry can be achieved using a sub-GHz frequency radio (specifically 400-900 MHz, including 433 MHz radios) communicating to a small receiver, such as a key fob-sized receiver that could be worn by the patient or otherwise placed on the patient's person that mediates data transfer between the ingested device and a smartphone via Bluetooth.

Examples of anxiety disorders include generalized anxiety disorder, social anxiety disorder (e.g. social phobia), specific phobias, separation anxiety disorder, post-traumatic stress disorder, or combinations thereof. The anxiety may also result from a medical condition that requires treatment. In the case of panic attacks/panic disorder, such conditions involve repeated episodes of sudden feelings of intense anxiety and fear or terror that reach a peak within minutes (panic attacks). The majority of the 13 known symptoms of panic attack are physiological: shortness of breath, heart racing, dizziness, chest pain, sweating, hot flashes, trembling, choking, nausea and numbness. Only three are not psychological: feeling of unreality, fear of losing control and fear of dying.

Each of the disclosed aspects and embodiments of the present disclosure may be considered individually or in combination with other aspects, embodiments, and variations of the disclosure. Further, while certain features of embodiments and aspects of the present disclosure may be shown in only certain figures or otherwise described in the certain parts of the disclosure, such features can be incorporated into other embodiments and aspects shown in other figures or other parts of the disclosure. Along the same lines, certain features of embodiments and aspects of the present disclosure that are shown in certain figures or otherwise described in certain parts of the disclosure can be optional or deleted from such embodiments and aspects. Additionally, when describing a range, all points within that range are included in this disclosure. Further, unless otherwise specified, none of the steps of the methods of the present disclosure are confined to any particular order of performance. Furthermore, all references cited herein are incorporated by reference in their entirety.

What is claimed is:

1. An anxiety monitor and treatment system comprising:
an ingestible housing;
at least one sensor configured to detect a physiological parameter indicative of anxiety; and
a controller operatively coupled to the least one sensor, the controller programmed to receive the physiological parameter and prepare the physiological parameter for transmission from the subject, analysis of the physiological parameter, or both.

2. The anxiety monitor and treatment system of claim 1, wherein the housing is configured to remain in the stomach for a period of time sufficient to allow the at least one sensor to detect the physiological parameter indicative of anxiety.

3. The system of claim 2, wherein the housing comprises detachable residency arms at a distal end thereof sized and configured to retain the housing in the stomach until the at least one sensor has detected a physiological parameter indicative of anxiety in the stomach.

4. The system of claim 1, further comprising a drug dispenser contained within housing and comprising an anti-anxiety medication.

5. The system of claim 4, wherein the drug dispenser is non-refillable.

6. The system of claim 4, wherein the controller is operably coupled to the drug dispenser.

7. The system of claim 4, wherein the controller is programmed to actuate release of the anti-anxiety medication from the drug dispenser into the patient in response to a determination that the physiological parameter falls outside a threshold value or range for the at least one physiological parameter.

8. The system of claim 7, wherein the controller is programmed to preemptively actuate release of the anti-anxiety medication from the drug dispenser into the patient in response to the determination such that the anti-anxiety medication is delivered prior to the patient experiencing the anxiety.

9. The system of claim 4, wherein detecting a physiological parameter indicative of anxiety comprises detecting an individual physiological parameter, a combination of physiological parameters, trends in an individual physiological parameter, or trends in a plurality of physiological parameters.

10. The system of claim 9, wherein the controller is programmed to actuate release of the anti-anxiety medication from the drug dispenser into the patient in response to a determination that the individual physiological parameter, the combination of physiological parameters, trends in the individual physiological parameter, or trends in the plurality of physiological parameters meet a pre-defined condition.

11. The system of claim 10, wherein the controller is programmed to preemptively actuate release of the anti-anxiety medication from the drug dispenser into the patient in response to the determination such that the anti-anxiety medication is delivered prior to the patient experiencing the anxiety.

12. The system of claim 1, wherein the controller and at least one sensor are contained within the housing.

13. The system of claim 4, wherein the housing is configured to reside in the stomach for period of time sufficient to allow release of the anti-anxiety medication into the patient.

14. The system of claim 13, wherein the housing comprises detachable residency arms at a distal end thereof sized and configured to retain the housing in the stomach until release of the anti-anxiety medication into the patient.

15. The system of claim 1, wherein the at least one sensor is an accelerometer, a photoplethysograph (PPG) sensor, an impedance plethysmograph (IPG) sensor, an electrocardiogram (ECG) sensor, a temperature sensor, or combinations thereof.

16. The system of claim 1, wherein the at least one sensor is a transcutaneous sensor configured to measure electrical conductance of the skin.

17. The system of claim 1, further comprising a transcutaneous electrode configured to deliver therapeutic electrical stimulation in response to the detected physiological parameter.

18. The system of claim 1, further comprising a radio configured to communicate with an external device, wherein the controller is configured to control the operation of the radio to send an alert to the external device in response to determining that the subject is experiencing anxiety.

19. The system of claim 1, further comprising a radio configured to communicate with an external device, wherein the controller is configured to control the operation of the radio to preemptively send an alert to the external device in response to receiving the physiological parameter such that alert is sent prior to the subject experiencing anxiety.

20. The system of claim 1, wherein the anxiety is a panic attack.
